(19) United States
(12) Patent Application Publication
(10) Pub. No.: US 2023/0056880 A1
(43) Pub. Date: Feb. 23, 2023

(54) PRESSING POSITION AND PRESSURE MEASUREMENT METHOD BASED ON PHOTOPLETHYSMOGRAPHIC IMAGING

(71) Applicant: ZHEJIANG LAB, Hangzhou (CN)

(72) Inventors: Ruofan WANG, Hangzhou (CN); Chiye LI, Hangzhou (CN); Junhui SHI, Hangzhou (CN)

(21) Appl. No.: 17/979,766

(22) Filed: Nov. 3, 2022

Related U.S. Application Data
(63) Continuation of application No. PCT/CN2021/112208, filed on Aug. 12, 2021.

(51) Int. Cl. A61B 5/0295; A61B 5/00; A61B 5/11

(52) U.S. Cl. CPC A61B 5/0295; A61B 5/0077; A61B 5/1128

(57) ABSTRACT

A pressing position and pressure measurement method based on PPG (photoplethysmographic) imaging, which only needs a camera to locate multiple pressing areas and measure corresponding pressure values without a pressure sensor. Before the measurement starts, only a simple calibration work is needed. With the gradual increase of the pressing pressure, the characteristics of PPG signals corresponding to diastolic pressure and systolic pressure will disappear one by one, and by recording two sets of pressure values and the corresponding PPG signal intensity values, the relation curve of the pressure versus blood perfusion changes can be fitted. Through this relation curve, the pressure values corresponding to different blood PPG signal intensities can be obtained. Different from the traditional technical route, the present disclosure provides a non-contact measurement method for determining the pressing location and measuring the pressure through a camera.

### CROSS-REFERENCE TO RELATED APPLICATIONS

The present application is a continuation of International Application No. PCT/CN2021/112208, filed on Aug. 12, 2021, the contents of both of which are incorporated herein by reference in their entireties.

### TECHNICAL FIELD

The present disclosure belongs to the field of machine vision analysis of limb PPG (photoplethysmographic) signals, and particularly relates to a pressing position and pressure measurement method based on PPG imaging.

### BACKGROUND

In touch interactions, the realization of traditional pressure sensing depends on setting a specific pressure sensor on the pressing surface. The sensor device contains elements that produce a strain change upon a pressure, and the pressure can be sensed by measuring the corresponding parameters (such as resistance, capacitance, etc.) of the elements. When the absolute value of the pressure needs to be determined by the sensor, it is necessary to calibrate the sensor before use to calibrate the signal characteristics corresponding to the specific pressure values. Such calibration is time-consuming, labor-intensive, complicated, and may drift with time, resulting in inaccurate measurement.

If the spatial position of a touch point is to be located by the traditional pressing measurement technology, one or more sensor arrays are required, and the response characteristics of different sensor units in the arrays are required to have high consistency. The resulting problems are that the spatial resolution is usually low, the locating accuracy is low, and the complexity and manufacturing cost of the device will increase, which brings inconvenience to large-scale applications.

### SUMMARY

In view of the shortcomings of the existing pressure sensing interaction technology, the present disclosure provides a pressing position and pressure measurement method based on PPG imaging.

The purpose of the present disclosure is realized by the following technical solution: a pressing position and pressure measurement method based on PPG imaging, including:

(1) Placing a camera that covers a pressing area to be detected, collecting video by the camera, and measuring a blood perfusion change from a change of a PPG signal intensity of the pressing area to be detected in an image channel of each video frame, so as to measure a change of blood perfusion in tissue blood vessels in the pressing area of a human limb.

(2) Carrying out calibration before the measurement starts; touching the pressing area with a finger and gradually increasing a pressure; with the increase of the pressing pressure, obtaining PPG signal intensities under different pressures, so that the PPG signal characteristics of a diastolic pressure and a systolic pressure disappear sequentially; firstly, recording a corresponding PPG signal intensity v₁ and a diastolic pressure p₁ as the PPG signal characteristics of the diastolic pressure disappear; with the increase of the pressing pressure, recording a corresponding PPG signal intensity v₂ and a systolic pressure p₂ as the PPG signal characteristics of the systolic pressure disappear; obtaining a waveform change diagram of the PPG signal intensity in the pressing area. When the characteristics of the PPG signal intensities of the systolic pressure and the diastolic pressure disappear respectively, the corresponding diastolic pressure p₁ and systolic pressure p₂ are applied pressure values; completing the calibration.

(3) Fitting an approximate relation curve of the pressure versus the blood PPG change: according to the waveform change diagram of the PPG signal intensity in the pressing area obtained in step (2), determining a moment when different applied pressures are equivalent to the diastolic pressure and the systolic pressure; according to the corresponding relation between the two groups of PPG signals obtained in step (2) and the applied pressures, obtaining by fitting the approximate relation curve of the change of the PPG signals versus the applied pressure.

(4) Locating the pressing position and measuring the pressure, and obtaining the pressing position and the applied pressure values according to the approximate relation curve of the change of the PPG signals versus the applied pressure obtained in step (3).

Further, the relation between the change of the PPG signals and the applied pressure in step (3) is determined by a compliance.

Further, the step (4) specifically comprises: identifying pressing areas of multi-point pressing by a camera, applying the relation curve of the applied pressure versus the change of a blood PPG signal intensity, and obtaining each pressing position and the pressure value corresponding to each pressing position according to the relation curve.

The present disclosure has the beneficial effects that, unlike the traditional technical route, the present disclosure provides a non-contact measurement method for determining the pressing position and measuring the pressure through the camera. With the simple method, a plurality of pressing areas can be flexibly and conveniently measured on a surface of any object without installing pressure sensors. Only a simple calibration work is required before starting the measurement in the present disclosure.

### BRIEF DESCRIPTION OF DRAWINGS

FIG. 1 is a schematic diagram of the positional relationship between the camera and the pressing area to be detected in the pressing position and measurement method of the present disclosure.

FIG. 2 (a) is a schematic diagram of the relation between a PPG signal intensity and time change of the present disclosure without pressing.

FIG. 2 (b) is a waveform change of a PPG signal intensity during the calibration process of the present disclosure.

FIG. 3 is a curve model fitted with data points obtained by simple calibration, showing the relation between the pressure measurement value of the present disclosure and the change of the blood PPG signal intensity.

### DESCRIPTION OF EMBODIMENTS

Photoplethysmography (PPG) is an optical measurement technology of biomedical tissues. After passing through human tissues, light with a specific wavelength will be absorbed and scattered by the tissues, resulting in attenuation. Through the attenuated optical signal, the change of the blood volume in tissue blood vessels can be monitored. Photoplethysmography records the absorption of light by the tissues of the part to be measured (e.g. finger tip, nasal valve) in real time, so as to obtain the blood volume of the blood vessels around the part and its pulsating change produced with the heartbeat. The photoplethysmography is widely used in the fields of physiological detection and diagnosis and treatment of cardiovascular diseases.

The present disclosure discloses a pressing position and pressure measurement method based on PPG imaging, which specifically includes:

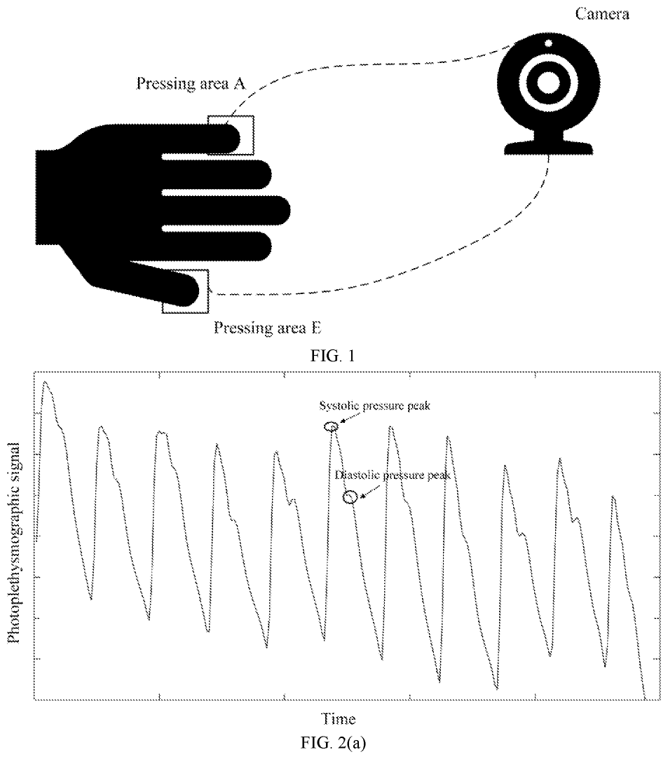

(1) A camera is placed to cover a pressing area to be detected, a video is collected by the camera, and a blood perfusion change of a PPG signal intensity of the pressing area to be detected in RGB channels of each frame image of the video is collected by photoplethysmography to measure a PPG change, so as to reflect the change of the limb pressing pressure through the change of a blood flow pressure in tissue blood vessels of the pressing area of the human body. The schematic diagram of the position relation between the camera and the pressing area to be detected in the present disclosure is shown in FIG. 1. The camera faces the pressing area to be detected, and the pressing position is determined by photographing the pressing action of the finger, and the changes of PPG signals in the finger area are collected.

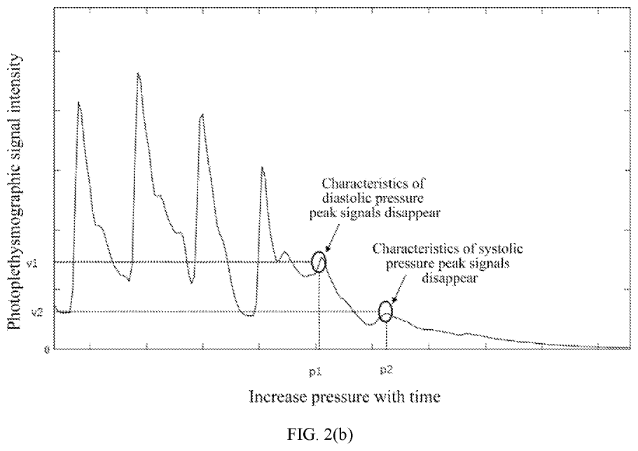

The blood volume near the pressing area of the human body is influenced by the blood pressure in the blood vessel and the outer applied pressure. When the finger exerts pressures of different intensities, the blood volume near this area will change in different degrees. As shown in FIG. 2 (a), it is a schematic diagram of a PPG signal of the pressing area A in FIG. 1 changing with time. Under the condition of no applied pressure, the change of PPG signal shows the change of the blood pressure near the pressing area of the human body caused by heartbeat. The peak of the signal indicates that the pressure in the blood vessel at this moment is a systolic pressure, which is marked as a systolic pressure peak in FIG. 2(a), and the second maximum peak formed over time is a diastolic pressure peak.

(2) Calibration is carried out before the measurement starts; by taking the pressing area A in FIG. 1 as an example, the pressing area is touched with a finger and a pressure is gradually increased; with the increase of the pressing pressure, PPG signal intensities under different pressures are obtained; after the pressure reaches a certain value, the characteristics of the PPG signals of a diastolic pressure and a systolic pressure disappear sequentially. First, a corresponding PPG signal intensity v₁ and a diastolic pressure p₁ are recorded as the characteristics of the PPG signal of the diastolic pressure disappear; with the increase of the pressing pressure, a corresponding PPG signal intensity v₂ and a systolic pressure p₂ are recorded as the characteristics of the PPG signal of the systolic pressure disappear. The diastolic pressure p₁ and systolic pressure p₂ can be obtained by instrument measurement or approximated by the average values of people. The diastolic pressure p₁ and systolic pressure p₂ are the values of the applied pressure. A waveform change diagram of the PPG signal intensity in the pressing area A is obtained, as shown in FIG. 2(b). When the characteristics of the PPG signal intensities of the systolic pressure and the diastolic pressure disappear respectively, the corresponding applied pressure value is the value of the equivalent blood pressure at this moment. The calibration is completed.

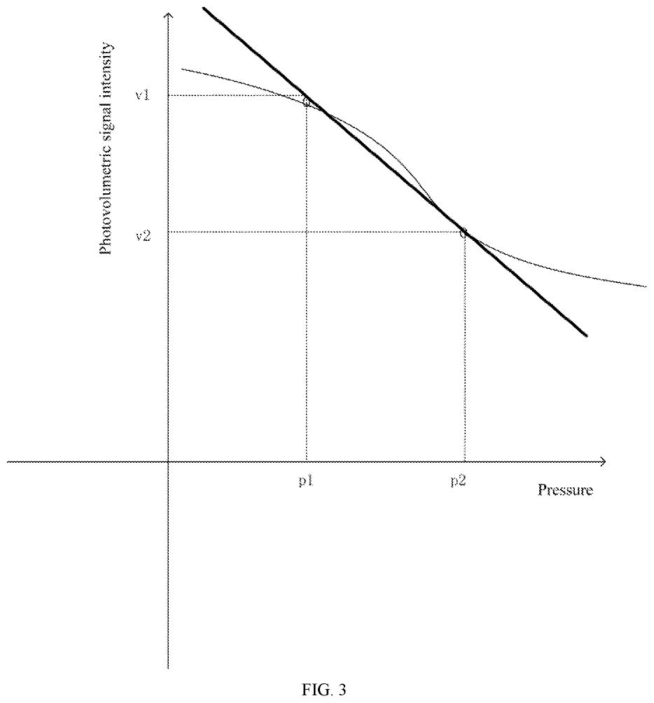

(3) An approximate relation curve of the pressure versus the blood perfusion change is fitted: according to the waveform change diagram of the PPG signal intensity in the pressing area as shown in FIG. 2(b), the changes in morphological characteristics of the blood pressure peaks in the PPG signals are used to estimate the pressing pressure, and the moments when different applied pressures are equivalent to the diastolic pressure and the systolic pressure can be determined respectively. According to the corresponding relationship between the two groups of PPG signals and the applied pressures, the relation curve of the PPG signal intensity versus the pressure can be obtained. The relation between the PPG signal intensity and the applied pressure is determined by a compliance, and the compliance of a blood vessel can be considered constant under the condition of a small tissue deformation amount within a small pressure application range, and the blood PPG signal intensity and the pressure have a linear relationship under the approximate condition. The approximate relation curve of the PPG signal intensity versus the applied pressure can be fitted by using the two groups of data points (p₁, v₁) and (p₂, v₂) obtained in Step 4, as shown by the bold straight line in FIG. 3. Through the approximate relation curve of the PPG signal intensity versus the applied pressure, the applied pressure values corresponding to different blood PPG signal intensities can be obtained.

(4) Locating the pressing position and measuring the pressure: the camera and the pressing area to be detected are arranged according to step (1), and a blood perfusion change is measured from a change of the PPG signal intensity of the pressing area to be detected in an image channel of each frame of the video collected by photoplethysmography. In the use process, multiple pressed limb parts can be identified by the camera, and multi-point pressing perception identification can be carried out. The relation curve of the applied pressure is applied versus the change of the blood PPG signal intensity, so the value of the applied pressure in each pressing area can be measured at the same time. The pressing position and the applied pressure value are obtained according to the approximate relation curve of the PPG signal intensity versus the applied pressure obtained in step (3).

To sum up, different from the traditional technical route, the present disclosure proposes a non-contact measurement method for determining the pressing position and measuring the pressure through a camera. The method is simple and can flexibly and conveniently measure a plurality of pressing areas on the surface of any object without installing a pressure sensor.

What is claimed is:

1. A pressing position and pressure measurement method based on PPG (photoplethysmographic) imaging, comprising:
step (1): placing a camera that covers a pressing area to be detected, collecting video by the camera, and measuring a blood perfusion change from a change of a PPG signal intensity of the pressing area to be detected in an image channel of each video frame, so as to measure a change of a blood perfusion in a tissue blood vessel in the pressing area of a human limb;
step (2): carrying out calibration before the measurement starts; touching the pressing area with a finger and gradually increasing a pressure; with the increase of the pressing pressure, obtaining PPG signal intensities under different pressures, so that the PPG signal characteristics of a diastolic pressure and a systolic pressure disappear sequentially; firstly, recording a corresponding PPG signal intensity v₁ and a diastolic pressure p₁ as the PPG signal characteristics of the diastolic pressure disappear; with the increase of the pressing pressure, recording a corresponding PPG signal intensity v₂ and a systolic pressure p₂ as the PPG signal characteristics of the systolic pressure disappear; obtaining a waveform change diagram of the PPG signal intensity in the pressing area, wherein when the characteristics of the PPG signal intensities of the systolic pressure and the diastolic pressure disappear, respectively, the corresponding diastolic pressure p₁ and systolic pressure p₂ are applied pressure values; completing the calibration;
step (3): fitting an approximate relation curve of the pressure versus the blood perfusion change: according to the waveform change diagram of the PPG signal intensity in the pressing area obtained in step (2), determining a moment when different applied pressures are equivalent to the diastolic pressure and the systolic pressure; according to the corresponding relation between the two groups of PPG signals obtained in step (2) and the applied pressures, obtaining by fitting the approximate relation curve of the change of the PPG signals versus the applied pressure; and
step (4): locating the pressing position and measuring the pressure, and obtaining the pressing position and the applied pressure values according to the approximate relation curve of the change of the PPG signals versus the applied pressure obtained in step (3).

2. The pressing position and pressure measurement method based on PPG imaging according to claim 1, wherein the relation between the change of the PPG signals and the applied pressure in step (3) is determined by a compliance.

3. The pressing position and pressure measurement method based on PPG imaging according to claim 1, wherein the step (4) specifically comprises: identifying pressing areas of multi-point pressing by a camera, applying the relation curve of the applied pressure versus the change of a blood PPG signal intensity, and obtaining each pressing position and the pressure value corresponding to each pressing position according to the relation curve.
